(19) United States
(12) Patent Application Publication
(10) Pub. No.: US 2018/0085027 A1
(43) Pub. Date: Mar. 29, 2018

(54) MR COMPATIBLE PUNCTURE CATHETER

(71) Applicant: Imricor Medical Systems, Inc., Burnsville, MN (US)

(72) Inventors: Scott KIMMEL, Roseville, MN (US); Steven R. Wedan, Savage, MN (US); Thomas W. Lloyd, Eagan, MN (US); Nicholas J. Kampa, Eagan, MN (US)

(21) Appl. No.: 15/567,514

(22) PCT Filed: Apr. 27, 2016
(86) PCT No.: PCT/US16/29499

§ 371 Date: Oct. 18, 2017

Related U.S. Application Data
(60) Provisional application No. 62/153,829, filed on Apr. 28, 2015.
(60) Provisional application No. 62/232,902, filed on Sep. 25, 2015.

(51) Int. Cl. A61B 5/06; A61M 25/01; A61M 25/06; A61B 5/04; A61M 25/00; G01R 33/28

(52) U.S. Cl. CPC A61B 5/062; A61M 25/0127; A61M 25/06; A61B 5/04; A61B 5/055; G01R 33/287; A61M 2025/0166; A61M 2205/3303; A61M 25/0084

(57) ABSTRACT

An MR compatible injection catheter is provided. The MR compatible injection catheter includes an inner shaft; an outer shaft circumferentially surrounding the inner shaft; and a means for actively tracking the catheter in a patient within a MRI. The means for actively tracking the catheter includes two or more tracking coils in the outer shaft. The inner shaft is configured to move relative to the outer shaft and includes an inner tube circumferentially surrounded by an outer tube.

### FIELD OF THE INVENTION

This invention relates to deflectable medical catheters. More particularly, this invention is related to medical injection catheters.

### BACKGROUND OF THE INVENTION

Traditionally, deflectable medical catheters have been used in interventional procedures to deliver therapies, such as RF energy, or implantables, such as leads or valves, into the body. Medical catheters have also been used for imaging and diagnostic purposes. Additionally, medical catheters, such as those with balloons, have been used to modify a patient's anatomy, such as during a structural heart application. An emerging catheter-based therapy is the delivery of liquids into tissue. An example of such a therapy is chemo-ablation, which is the destruction of cells via delivery of ethanol or a similar cytotoxic liquid into tissue. Chemo-ablation could be used to replace RF ablation for arrhythmia modification or for targeted chemotherapy of tumors. Another example is stem cell therapy, in which a solution containing stem cells and supporting liquids is delivered into the tissue to replace damaged cells. The aforementioned therapies require a precise method for delivering the solutions into the target tissue. This precision is predicated upon detailed tissue visualization and accurate navigation of the catheter tip to the target tissue location.

MRI has achieved prominence as a diagnostic imaging modality, and increasingly as an interventional imaging modality. The primary benefits of MRI over other imaging modalities, such as X-ray, include superior soft tissue imaging and avoiding patient exposure to ionizing radiation. The superior capability for imaging soft tissue using MRI has offered great clinical benefit with respect to diagnostic imaging. Similarly, interventional procedures, which have traditionally used X-ray imaging for guidance, stand to benefit greatly from soft tissue imaging only available with MRI. In addition, the significant patient exposure to ionizing radiation associated with traditional X-ray guided interventional procedures is eliminated with MRI guidance.

MRI uses three fields to image patient anatomy: a large static magnetic field, a time-varying magnetic gradient field, and a radiofrequency (RF) electromagnetic field. The static magnetic field and time-varying magnetic gradient field work in concert to establish both proton alignment with the static magnetic field and also spatially dependent proton spin frequencies (resonant frequencies) within the patient. The RF field, applied at the resonance frequencies, disturbs the initial alignment, such that when the protons relax back to their initial alignment, the RF emitted from the relaxation event may be detected and processed to create an image.

Each of the three fields associated with MRI presents safety risks to patients when a medical device is in close proximity to or in contact either externally or internally with patient tissue. One important safety risk is the heating that may result from an interaction between the RF field of the MRI scanner and the medical device (RF-induced heating), especially medical devices that have elongated conductive structures, such as braiding and pull-wires in catheters and sheaths.

The RF-induced heating safety risk associated with elongated metallic structures in the MRI environment results from a coupling between the RF field and the metallic structure. In this case several heating related conditions exist. One condition exists because the metallic structure electrically contacts tissue. RF currents induced in the metallic structure may be delivered into the tissue, resulting in a high current density in the tissue and associated Joule or Ohmic tissue heating. Also, RF induced currents in the metallic structure may result in increased local specific absorption of RF energy in nearby tissue, thus increasing the tissue's temperature. The foregoing phenomenon is referred to as dielectric heating. Dielectric heating may occur even if the metallic structure does not electrically contact tissue, such metallic braiding used in a deflectable sheath. In addition, RF induced currents in the metallic structure may cause Ohmic heating in the structure, itself, and the resultant heat may transfer to the patient. In such cases, it is important to attempt to both reduce the RF induced current present in the metallic structure and/or eliminate it all together by eliminating the use of metal braid and long metallic pull-wires.

The static field of the MRI will cause magnetically induced displacement torque on any device containing ferromagnetic materials and has the potential to cause unwanted device movement. It is important to construct the sheath and control handle from non-magnetic materials, to eliminate the risk of unwanted device movement.

When performing interventional procedures under MRI guidance, clinical grade image quality must be maintained. Conventional catheters and sheaths are not designed for the MRI and may cause image artifacts and/or distortion that significantly reduce image quality. Constructing the catheter from non-magnetic materials and eliminating all potentially resonant conductive structures allows the catheter to be used during active MR imaging without impacting image quality. Similarly, it is as important to ensure that the catheter control handle is also constructed from non-magnetic materials thereby eliminating potentially resonant conductive structures that may prevent the control handle being used during active MR imaging.

While there are many types of surgical instruments available, few are well-suited for use in an MRI environment. For example, deflectable (i.e., steerable) catheters and sheaths including multi-directional, bi-directional and uni-directional deflectable devices are known. However, many of these devices have ferromagnetic components that can result in undesired movement and a potential for patient injury, when placed in the strong magnetic field associated with MRI. The ferromagnetic components can also cause image distortions, thereby compromising the effectiveness of the procedure. Still further, such devices may include metallic components that may cause radiofrequency (RF) deposition in adjacent tissue and, in turn, tissue damage due to an extensive increase in temperature.

Conventional puncture and injection catheters have the same limitations. They either include ferromagnetic components that cause image distortion or include metallic components that cause RF deposition in tissue.

Moreover, it is difficult or impossible to track or visualize the location of the aforementioned devices in an MRI environment. In general, there are two types of device tracking useful for MRI guidance of interventional devices: active tracking and passive tracking. Active tracking is more robust and often preferable to passive tracking. However, active tracking is more difficult to implement in interventional devices and typically involves resonant RF coils that are attached to the device and directly connected to an MR receiver. These RF coils act as receive coils and allow for the determination of the three-dimensional coordinates of the coils within the scanner. To the inventors' knowledge neither active nor passive tracking techniques are presently utilized in conventional puncture/injection catheters.

Even with limited MRI compatible interventional devices currently available, a variety of MRI techniques are being developed as alternatives to X-ray imaging for guiding interventional procedures. For example, as a medical device is advanced through the patient's body during an interventional procedure, it is now possible to track its position so that the device can be visualized and delivered properly to a target site. Once delivered to the target site, the device and patient tissue can be monitored to improve therapy delivery. Thus, tracking the position of medical devices is useful in interventional procedures. Exemplary interventional procedures include, for example, cardiac electrophysiology procedures including diagnostic procedures for diagnosing arrhythmias and ablation procedures such as atrial fibrillation ablation, ventricular tachycardia ablation, atrial flutter ablation, Wolfe Parkinson White Syndrome ablation, AV node ablation, SVT ablations and the like. Tracking the position of medical devices using MRI is also useful in oncological procedures such as breast, liver and prostate tumor ablations; and urological procedures such as uterine fibroid and enlarged prostate ablations.

Thus, what is needed is an MR compatible puncture catheter so MR imaging can be utilized to ensure the precise delivery of therapeutic solutions into the target tissue. Such a device needs to be safe for use under MR guidance and allow for MR guidance and visualization.

### BRIEF SUMMARY OF THE INVENTION

In a first aspect of the MR compatible injection catheter in accordance with the invention, the injection catheter broadly includes an inner shaft; an outer shaft circumferentially surrounding said inner shaft; and a means for actively tracking the catheter in a patient within a MRI.

In another aspect of the invention the inner shaft slides within the outer shaft. The inner shaft is a long hollow tube that may consist of a braided catheter construction or a simple polymer extrusion. A puncture tip is operably connected to the distal tip of the inner shaft. The puncture tip includes a small, short cannula fixedly attached to the distal tip of the inner shaft. The cannula extends is a hollow tube that has a sharpened tip. The cannula is similar in shape to the distal tip section of a traditional transseptal needle. The connection between the puncture tip and the inner shaft is such that the lumen of the cannula is continuous with the lumen of the inner shaft. The inner diameter of the cannula lumen is preferably smaller than the inner diameter of the inner shaft lumen, but they could be the same size, or the lumen of the inner shaft could be smaller than the lumen of the cannula. The cannula could be constructed of metallic materials such as aluminum, inconel, nitinol, gold, etc. or of non-metallic materials such as PEEK, ceramic, zirconia, delrin, epoxy, etc. The puncture tip also contains a tracking coil and an O-ring. The distal end of the cannula terminates in a sharpened puncture tip.

The outer shaft is a long hollow tube that may consist of a braided catheter construction or a simple polymer extrusion. At the distal tip of the outer shaft is a tip support that includes a tracking coil. The tip support is preferably made of a non-metallic material such as PEEK, ceramic, zirconia, delrin, fiber reinforced epoxy, etc.

The inner shaft slides in relation to the outer shaft from a first retracted, proximal position to an extended distal position. In the retracted position, the puncture tip is completely housed within the tip support of the outer shaft. In the extended position, the cannula of the puncture tip is exposed and extends from the distal tip of the puncture catheter.

At the proximal end of the puncture catheter is a control handle. The control handle contains an advancement mechanism that allows the clinician to advance the inner shaft, which moves the puncture tip into the extended position. The advancement mechanism could be a sliding button, a control knob, etc. The lumen of the inner shaft terminates on the proximal end in the control handle. At this end there is an opening that allows for injection of solution into the inner shaft lumen. The opening could be a simple luer fitting, hemostasis valve, or stopcock assembly as is common in medical catheters.

As mentioned above, there are two tracking coils, one on the outer tip support and one on the puncture tip. These two tracking coils allow the puncture catheter to be navigated to the target tissue location under MR guidance. During the navigation step, the puncture tip is in the retracted position, which ensures that the sharp cannula tip doesn't damage any tissue. When the target tissue is reached, the clinician advances the inner shaft, which causes the puncture tip to move into the extended position and the cannula to penetrate the target tissue. Once the cannula is in the tissue, the clinician injects the therapy solution in the proximal opening of the inner shaft lumen. This solution then travels down the inner shaft lumen and enters the target tissue.

Each tracking coil is connected to a transmission line that travels the length of the respective shaft and exits at the control handle. In this aspect, the inner tracking coil moves with the inner shaft and the inner transmission line also moves. As a result, there may be some slack in the transmission line in the control handle to accommodate this movement. Alternatively, the termination of the tracking coil transmission line may move with the inner shaft eliminating the need for slack in the transmission line.

The advantage of locating one tracking coil on the puncture tip and the other tracking coil on the outer tip support is that the linear position of puncture tip in relation to the outer shaft can be precisely measured. This is because the space between the two tracking coils changes when the puncture tip goes from the retracted position to the extended position. With this configuration, the precise location of the tip of the cannula can be displayed to the clinician and the clinician can then use this information to control the depth of penetration of the cannula tip into the tissue. This would give the clinician a finer degree of control over the location and extent of solution delivery into the target tissue.

The purpose of the O-Ring on the puncture tip is to ensure that there is no fluid ingress between the puncture tip and the outer tip support, while still allowing the puncture tip to slide in relation to the outer tip.

Another advantage of this aspect is that because the cannula is fixedly attached to the puncture tip and will always be in a distal position in relation to the tracking coils, it can be fabricated out of metal, such as aluminum, inconel, nitinol, gold, etc. If any portion of the inner shaft that is located under either of the tracking coils were made out of metal, there is a potential for disruption or distortion of the tracking signal. Fabricating the cannula out of metal is advantageous because a metal tube can have a thinner wall than a nonmetallic tube and have superior strength and bending resistance. A thinner wall translates to a larger inner diameter for the cannula, which further translates to easier delivery of viscous solutions such as those that might be required for delivery of stem cells. Finally, it is easier to grind the tip of a metal tube into more complicated and sharper bevel shapes, which could reduce the puncture force required to penetrate the target tissue.

This aspect could be made deflectable by locating one or more pull wires in the wall of either or both of the inner shaft or the outer shaft. Utilizing a braided catheter shaft construction for one or both of the inner and outer shafts would mean that deflectable regions could be created by placing a lower durometer or softer material in the distal section of the shafts. The one or more pull wires would be connected in the control handle to a mechanism that would allow the clinician to deflect the distal tip of the puncture catheter. This mechanism could be a slide button, rotation knob, etc.

Those of skill in the art will recognize that alternative aspects achieving a similar purpose are possible. For instance, two tracking coils could be located in the outer shaft and both remain fixed. This would eliminate the need for an inner tip support and thus the inner shaft could be directly connected or bonded to the cannula section, which could still be constructed of a metallic material. The advantage of the design of this aspect is that it is simpler and easier to manufacture. The disadvantage is that the measurement of the distance between the two tracking coils remains fixed and therefore the extent of cannula extension, and the related amount of tissue penetration, cannot be measured by the tracking coil locations and displayed to the clinician.

In another aspect with fixed tracking coils on the outer shaft, the inner shaft could be made up of two coaxial tubes. Preferably, the outer tube would be made of a more rigid material such as ceramic or fiber-reinforced epoxy, while the inner tube would be made of a more flexible material, such as polyimide, PEBAX, grilamid, etc. A deflectable region within the rigid outer tube could be created by spiral-cutting, spine-cutting, etc. the tube in a short section near the distal tip of the tube. The inner tube is not spiral cut and therefore creates a continuous, solid inner lumen, which would contain the injected fluid. In other words, if the inner tube were not present, the fluid would escape through the channels in the outer tube created by the spiral cut. Those of skill in the art will appreciate that the tubes could be reversed such that the inner tube is the stiffer, spiral cut material, while the outer tube is the more flexible material. The advantage of constructing the inner shaft in this manner is that it simplifies the design by reducing the number of components in the inner shaft. It also allows for using stiffer materials to construct the inner shaft. Stiffer materials translate to more column strength which potentially translates to lower puncture force.

In a similar aspect, the inner shaft could be removable from the puncture catheter. To reintroduce the inner shaft, which could comprise an injection needle, into the injection catheter, the catheter could be outfitted with an inner lumen connected to a hemostasis valve located at the catheter handle.

In yet another aspect, inner shaft and puncture tip/cannula could remain in a fixed position in relation to the outer tip support and outer shaft. A sliding cannula cover piece could conceal the tip of the cannula while the catheter is being navigated to the target tissue. The sliding cannula cover piece could be biased in a position covering the tip of the cannula and a pull wire or some other mechanism could be used to expose the tip of the cannula for delivery of therapy.

The shortcomings of the present injection catheters are addressed by the device in accordance with the invention. The MR safety of the injection needle is provided by the materials from which the needle is constructed. The use on non-magnetic materials and limiting the use of conductive materials eliminates the risk associated with magnetic displacement force and RF heating associated with MR guided interventional procedures.

The MR safety of the MR tracking coils is provided by the construction of the tracking coil transmission line. One method for doing this is to incorporate transformers into the transmission line.

For puncture catheter with integrated electrodes, the MR safety of the electrodes is provided by the electrode wire assembly. Examples of electrode wire assemblies safe for use during MR imaging include those described in U.S. Pat. No. 8,588,934 and U.S. Pat. No. 8,588,938, which are hereby incorporated by reference in their entireties.

### BRIEF DESCRIPTION OF THE DRAWINGS

For a better understanding of the invention, and to show how the same may be carried into effect, reference will now be made, by way of example, to the accompanying drawings, in which:

FIG. 1 is a perspective view of the distal section of one aspect of the injection catheter in accordance with the invention.

FIG. 2A is a perspective view of the puncture tip of the injection catheter of FIG. 1 in the retracted position.

FIG. 2B is a perspective view of the puncture tip of the injection catheter of FIG. 1 in the extended position.

FIG. 3 is a perspective view of another aspect of the puncture catheter in accordance with the invention with the puncture tip in the extended position.

FIG. 4 is a perspective view of another aspect of the invention showing the distal tracking coil and the proximal tracking coil located in fixed positions on the outer shaft.

FIG. 5 is a side cutaway view of the distal section of the inner shaft showing the inner tube and outer tube.

FIG. 6 is a perspective view of the distal section of the inner shaft showing the spiral cut region of the outer tube.

FIG. 7 is a perspective view of the injection catheter in accordance with the invention showing the cannula cover concealing the sharpened puncture tip and the cannula cover being held in the distal position by biasing means.

FIG. 8 is a perspective view of the injection catheter in accordance with the invention depicting that when tension is placed on the pull wire, the biasing means compress, the cannula cover slides proximally, and the sharpened puncture tip is exposed.

FIG. 9A is a solid perspective view of the sliding cannula cover in the extended position.

FIG. 9B is a solid perspective view of the sliding cannula cover in the retracted position with the puncture tip exposed.

### DETAILED DESCRIPTION OF THE INVENTION

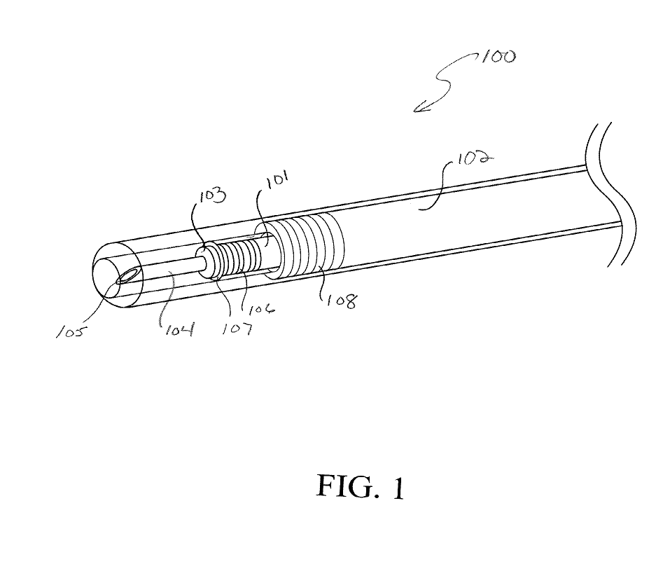

Referring now to the figures, FIG. 1 shows the distal end 100 of a first aspect of the invention. The injection catheter includes an inner shaft 101 that slides within an outer shaft 102. The inner shaft 101 is a long hollow tube that may consist of a braided catheter construction or a simple polymer extrusion. A puncture tip 103 is operably connected to the distal tip of the inner shaft 101. The puncture tip 103 has a small, short cannula 104 fixedly attached on its distal surface. The cannula extends distally from the puncture tip and is a hollow tube that has a sharpened tip 105. The cannula is similar in shape to the distal tip section of a traditional transseptal needle. The connection between the puncture tip 103 and the inner shaft 101 is such that the lumen of the cannula is continuous with the lumen of the inner shaft. The inner diameter of the cannula lumen is preferably smaller than the inner diameter of the inner shaft lumen, but they could be the same size, or the lumen of the inner shaft could be smaller than the lumen of the cannula. The cannula 104 could be constructed of metallic materials such as aluminum, inconel, nitinol, gold, etc. or of non-metallic materials such as PEEK, ceramic, zirconia, delrin, epoxy, etc. The puncture tip 103 also contains a tracking coil 106 and an O-ring 107. A second tracking coil 108 is located on the outer shaft. The injection catheter is made deflectable by locating one or more pull wires in the wall of either or both of the inner shaft or the outer shaft. Utilizing a braided catheter shaft construction for one or both of the inner and outer shafts would mean that deflectable regions could be created by placing a lower durometer or softer material in the distal section of the shafts. The one or more pull wires would be connected in the control handle to a mechanism that would allow the clinician to deflect the distal tip of the puncture catheter. This mechanism could be a slide button, rotation knob, etc.

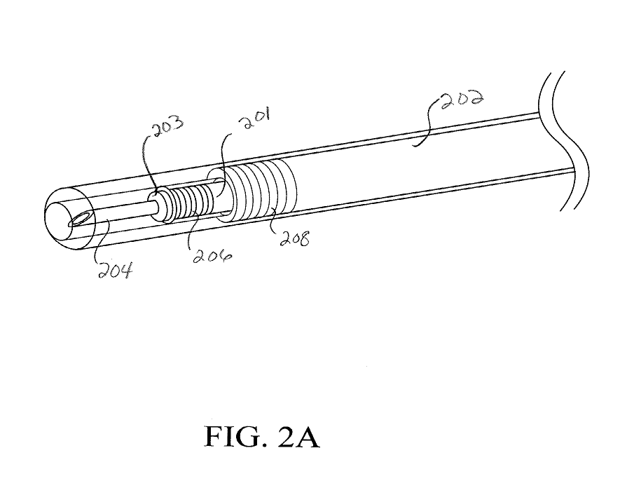

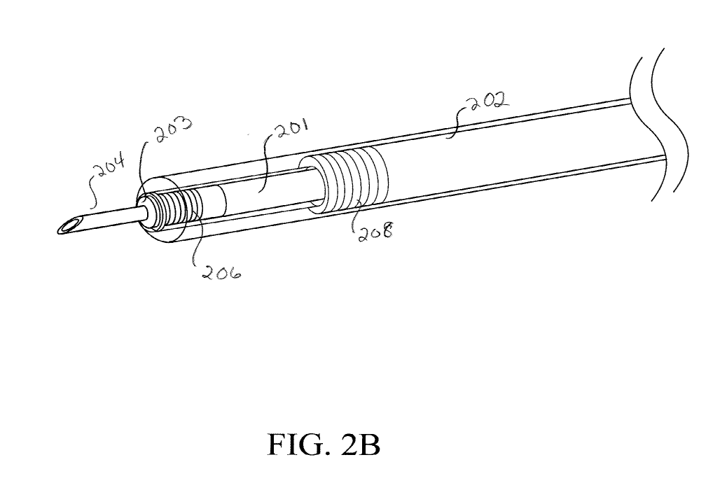

FIG. 2A shows the distal section of the injection catheter with the puncture tip 203 retracted. FIG. 2B shows the puncture tip 203 extended. In the retracted position, the tracking coil 206 on the puncture tip 203 is a fixed distance from the tracking coil 208 on the outer shaft 202. As the puncture tip 203 is extended, the distance between the tracking coil 203 on the puncture tip 203 and the tracking coil 208 on the outer shaft 202 increase by the distance the tip is extended. The distance between the tracking coils 206 and 208 provides information on the position of the puncture tip and location of the tip of the cannula 204. This information can be used to determine the precise location of the puncture tip and the degree to which it is extended. The position of the puncture tip can then be displayed during active tracking of the catheter.

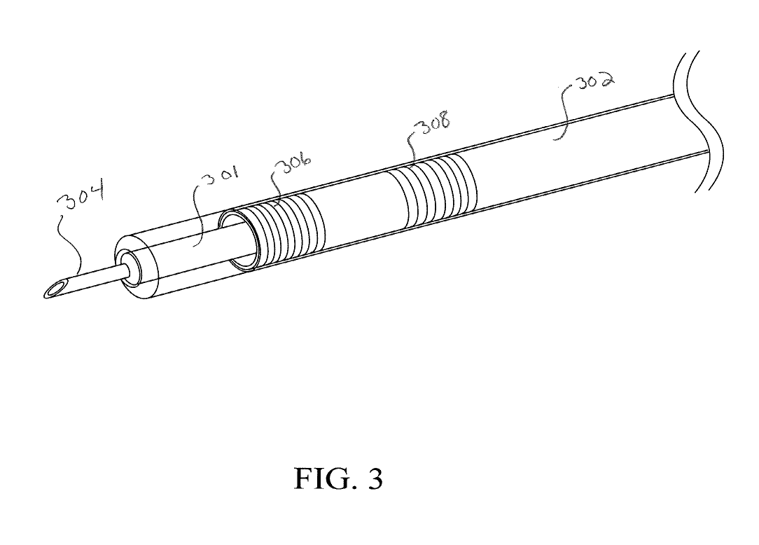

FIG. 3 shows an alternative aspect of the injection catheter. In this aspect, both tracking coils 306 and 308 are located on the outer shaft 302 and remain fixed in place on the outer shaft 302. This aspect removes the requirement for a puncture tip. This configuration allows the cannula 304 to be directly bonded to the inner shaft 301. This aspect of the injection catheter does not allow for precise determination of the cannula location as it is extended from the catheter. The advantage is a simplified manufacturing process and a reduction in the number of moving components within the catheter.

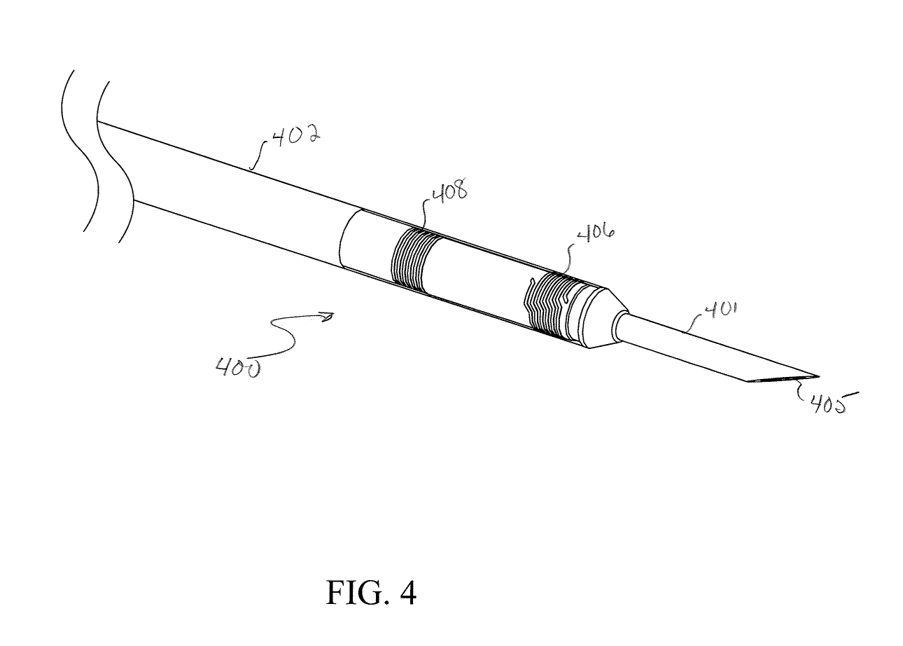

FIG. 4 shows a third aspect of the injection catheter 400 with the inner shaft 401 constructed in a different manner than the previous aspects. In this aspect, puncture catheter 400 has both the distal tracking coil 406 and the proximal tracking coil 408 located in fixed positions on the outer shaft 402. The inner shaft 401 is comprised of two coaxial tubes to form a single elongated cannula with an integrated sharpened tip 405. In such an aspect, the inner shaft 401 can be removable from the outer shaft 402.

The puncture catheter with a removable inner shaft includes a hemostasis valve and in the handle of the catheter that is connected to an inner lumen through which the inner shaft 401 can be inserted into the puncture catheter. The extent to which the sharpened tip 405 extends from the outer shaft 402 is controlled manually at the proximal end of the inner shaft 401 near the hemostasis valve where it enters the puncture catheter.

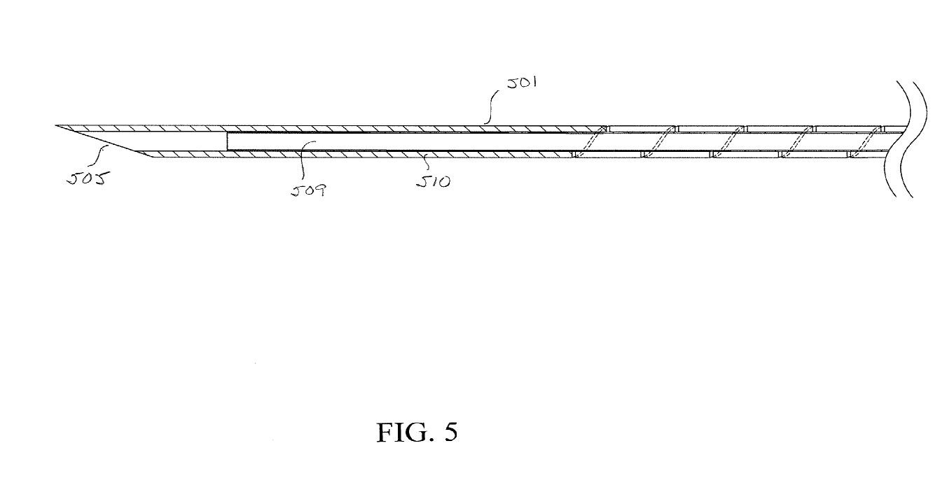

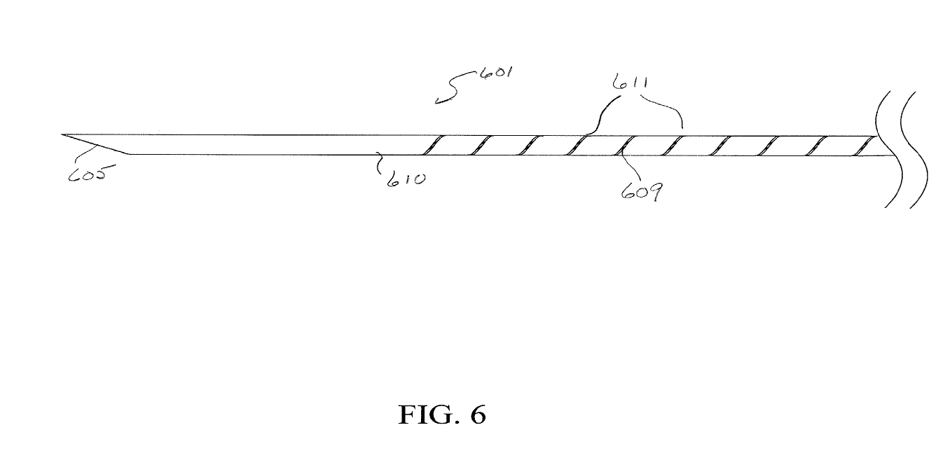

In reference to injection catheter 400, FIG. 5 depicts a cut-away side view of the inner tube 509 and the outer tube 510 comprising the inner shaft 501. The outer tube 510 is constructed of a rigid material such as ceramic or fiber-reinforced epoxy. The inner tube 509 is made from a flexible material such as polyimide, PEBAX, grilamid, etc. This construction allows for a portion of the rigid outer tube 510 to be made flexible while maintaining a continuous lumen for fluid delivery. This is illustrated in FIG. 6.

FIG. 6 shows the inner shaft 601 with a spiral cut 611 region of the outer tube 610. Cutting the outer tube 610 using a method such as spiral-cutting, spline-cutting, etc. creates a region of the outer tube 610 that can be deflected. The inner tube 609, which is only visible through the spiral cut 611 in the outer tube 610, forms a continuous inner lumen within the outer tube 610. As such, the inner tube 610 allows fluids to be delivered through the inner shaft 601. Those of skill in the art will appreciate that the inner tube 609 and the outer tube 610 could be reversed such that the inner tube 609 is the rigid material that is cut to create a deflectable region while the outer tube 610 is the more flexible material. Constructing the inner shaft 601 in this manner allows for use of a rigid material in construction of the inner shaft 601, which results in more column strength and increased transfer of force to the sharpened tip 605.

Although inner shaft described in FIGS. 4-6 is described as comprising two coaxial tubes. It is obvious to those of skill in the art that a removable inner shaft can be made of various constructions and is not limited to the specific construction disclosed.

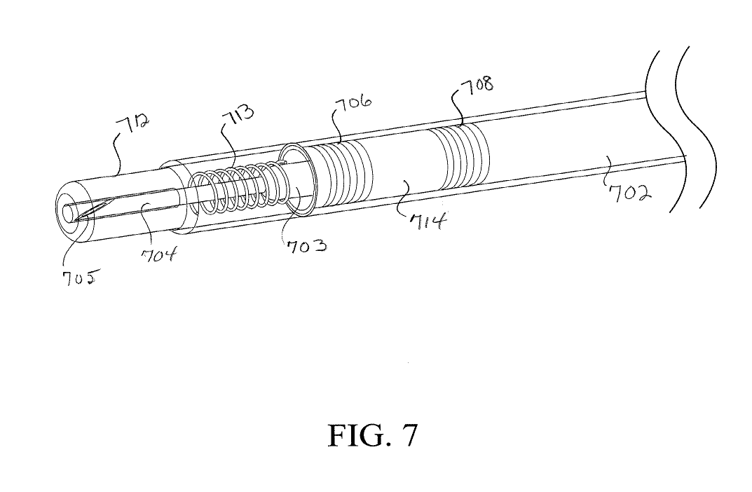

FIG. 7 shows another aspect of the invention where the inner shaft (not shown), puncture tip 703, and cannula 704 remain in a fixed position relative to two fixed tracking coils 706 and 708, and the outer shaft 702. In this aspect, the tip support 714 that holds the two tracking coils 706 and 708 has an inside diameter that is large enough to support an additional sliding cannula cover piece 712. The sliding cannula cover piece 712 functions to conceal the sharpened puncture tip 705 of the cannula 704 while the catheter is being navigated to the target tissue. Biasing means 713 biases the sliding cannula cover 712 in the distal position to cover the sharpened puncture tip 703. Biasing means 713 is shown as a compression spring but those of skill in the art will appreciate that any biasing means may be utilized.

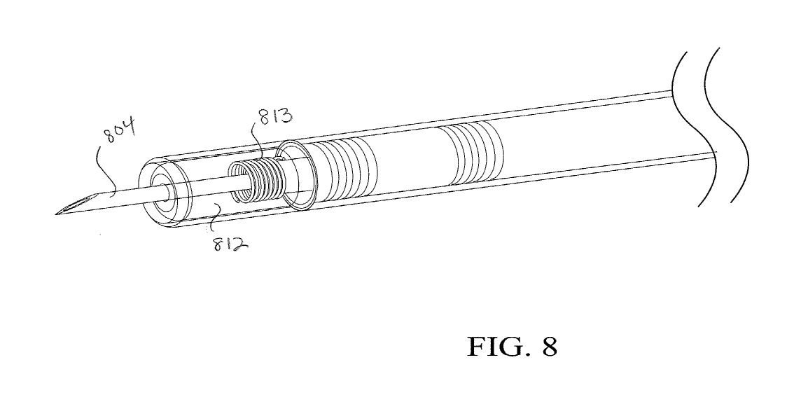

FIG. 8 shows the catheter with the cannula cover 812 retracted. This can be accomplished by means of a pull wire (not shown) with one end of the pull wire attached to the proximal edge of the cannula cover 812 and the other end of the pull wire connected to a retraction mechanism in the catheter handle such as rotation knob or sliding lever. When tension is placed on the pull wire, the sliding cannula cover 812 is pulled proximally, the spring 813 is compressed, and the cannula 804 is exposed. When tension is released on the pull wire, the spring 813 extends, and the cannula cover 812 extends and conceals the cannula 804.

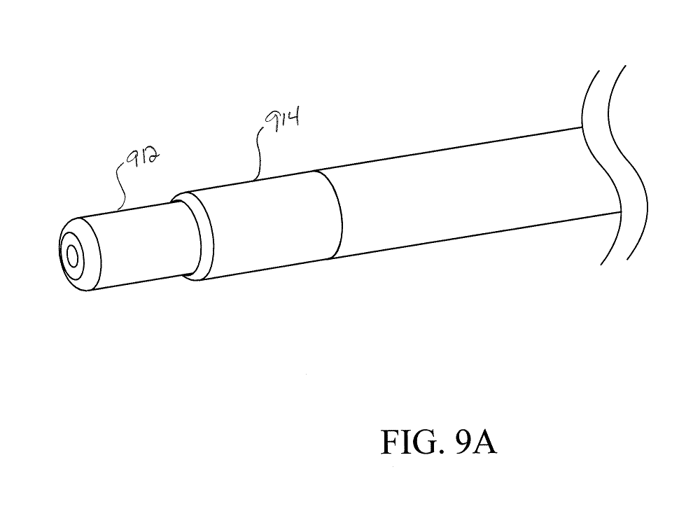

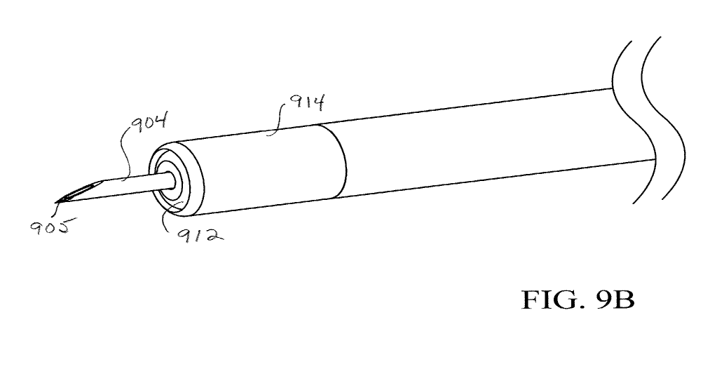

FIG. 9 provides a solid isometric view of an injection catheter with a retractable cannula cover 912. FIG. 9A shows the cannula cover 912 extended from the tip support 914. FIG. 9B shows the cannula cover 912 retracted into the tip support 914 with the sharpened tip 905 of cannula 904 exposed.

The injection catheter described herein can be made safe from the risk of magnetic displacement force by use of non-magnetic materials. The risk of RF heating can be eliminated by limiting conductive materials to the puncture tip, RF safe electrode lines, and transmission lines with integrated transformers.

To facilitate electrophysiological measurements during an interventional procedure, the injection catheter can include one or more electrodes. To achieve RF safety of the electrodes, RF safe electrode lines such as those described in U.S. Pat. No. 8,588,934 and U.S. Pat. No. 8,588,938 may be used to connect the electrodes to a connector in the catheter handle.

In all of the various aspects of the injection catheter disclosed herein, two tracking coils are shown; however, those of skill in the art will appreciate that the invention is not necessarily limited to two tracking coils. Rather injection catheters utilizing only one tracking coil are also intended to fall within the scope of the invention.

Although the present invention has been described with reference to various aspects of the invention, those of ordinary skill in the art will recognize that changes may be made in form and detail without departing from the spirit and scope of the invention.

What is claimed is:

1. An MR compatible injection catheter comprising:
an inner shaft;
an outer shaft circumferentially surrounding said inner shaft; and
a means for actively tracking the catheter in a patient within a MRI.

2. The MR compatible injection catheter of claim 1 wherein said means for actively tracking the catheter in a patient within a MRI comprises at least one tracking coil.

3. The MR compatible injection catheter of claim 1 wherein said means for actively tracking the catheter in a patient within a MRI comprises two or more tracking coils.

4. The MR compatible injection catheter of claim 3 wherein spacing between the two or more tracking coils is configured to vary with the position of the injection needle or cannula.

5. The MR compatible injection catheter of claim 4 wherein the spacing between the two or more tracking coils is configured to determine the location of the puncture tip.

6. The MR compatible injection catheter of claim 1 further comprising one or more electrodes configured to perform one or more electrophysiology measurements.

7. The MR compatible injection catheter of claim 1 wherein the inner shaft is configured to move relative to the outer shaft.

8. The MR compatible injection catheter of claim 1 wherein the inner shaft comprises an inner tube circumferentially surrounded by an outer tube.

9. The MR compatible injection catheter of claim 8 wherein the outer tube includes a spirally-cut deflectable portion.

10. The MR compatible injection catheter of claim 8 wherein said spirally-cut deflectable portion comprises a stiff material.

11. The MR compatible injection catheter of claim 8 wherein said inner tube comprises a flexible material.

12. The MR compatible injection catheter of claim 11 wherein said flexible material is selected from grilamid, polyimide, and PEBAX.

13. The MR compatible catheter of claim 10 wherein said stiff material is selected from fiber reinforced epoxy, ceramic, and liquid crystal polymer.

14. The MR compatible injection catheter of claim 8 wherein a tip of said outer tube is beveled to from a sharpened, puncture tip.

15. The MR compatible injection catheter of claim 7 wherein a distal end of the inner shaft is operably coupled to a cannula section having a sharpened tip.

16. The MR compatible injection catheter of claim 7 wherein the inner shaft includes a puncture tip operably coupled thereto.

17. The MR compatible injection catheter of claim 16 wherein said puncture tip is bonded to said inner shaft.

18. The MR compatible catheter of claim 17 wherein said puncture tip includes a tracking coil.

19. The MR compatible catheter of claim 18 wherein the puncture tip includes a cannula section having a sharpened tip.

20. The MR compatible catheter of claim 19 wherein the cannula is constructed from a material selected from metal, ceramic, PEEK, and fiber-reinforced epoxy.

21. The MR compatible catheter of claim 1 wherein the inner shaft is fixed relative to the outer shaft.

22. The MR compatible catheter of claim 21 wherein the inner shaft includes a puncture tip operably coupled thereto.

23. The MR compatible catheter of claim 22 further comprising a cannula cover slidable between a first extended position and a second retracted position.

24. The MR compatible catheter of claim 23 wherein said cannula cover is biased in the first extended position by biasing means.

25. The MR compatible catheter of claim 24 wherein said biasing means comprises a compression spring.

26. The MR compatible catheter of claim 23 further comprising a pull wire operably coupled to the cannula cover and configured to cause said cannula to slide the second retracted position.

27. The MR compatible catheter of claim 22 wherein the means for actively tracking the catheter comprises two or more tracking coils in the outer shaft.

28. The MR compatible catheter of claim 7 wherein the means for actively tracking the catheter comprises two or more tracking coils in the outer shaft.

29. The MR compatible catheter of claim 28 wherein the inner shaft comprises a polymer extrusion and the tip is coupled to a cannula section having a sharpened tip.

30. The MR compatible catheter of claim 27 wherein said polymer comprises Grilamid, PEEK or polyimide.

31. The MR compatible catheter of claim 29 wherein said cannula is constructed of metal.

32. The MR compatible catheter of claim 31 wherein said metal is Elgiloy, Nitinol, MP35N, Titanium, Stainless Steel, or Tungsten.

33. The MR compatible catheter of claim 29 wherein said cannula is constructed of a non-metallic material such as ceramic or fiber-reinforced epoxy.

34. The MR compatible catheter of claim 8 wherein the inner tube includes a spirally-cut deflectable portion.

35. The MR compatible injection catheter of claim 34 wherein said spirally-cut deflectable portion comprises a stiff material.

36. The MR compatible injection catheter of claim 34 wherein said stiff material is selected from fiber reinforced epoxy, ceramic, and liquid crystal polymer.

37. The MR compatible injection catheter of claim 34 wherein said outer tube comprises a flexible material.

38. The MR compatible injection catheter of claim 37 wherein said flexible material is selected from grilamid, polyimide, and PEBAX.

39. The MR compatible injection catheter of claim 34 wherein a tip of said inner tube is beveled to from a sharpened, puncture tip.
